(19) United States
(12) Patent Application Publication
(10) Pub. No.: US 2025/0072786 A1
(43) Pub. Date: Mar. 6, 2025

(54) VIBRATIONAL SYSTEM FOR SUBJECT PROPRIOCEPTIVE IMPAIRMENT STUDY

(71) Applicant: UNIVERSITY OF SOUTH CAROLINA, COLUMBIA, SC (US)

(72) Inventors: DEBDYUTI MANDAL, CAYCE, SC (US); SOURAV BANERJEE, IRMO, SC (US); JOHN R. GILLIAM, COLUMBIA, SC (US); SHERI SILFIES, MOORESVILLE, NC (US)

(21) Appl. No.: 18/813,126

(22) Filed: Aug. 23, 2024

Related U.S. Application Data
(60) Provisional application No. 63/535,696, filed on Aug. 31, 2023.

(51) Int. Cl. A61B 5/11; A61B 5/316; G06F 3/0484

(52) U.S. Cl. CPC A61B 5/1107; A61B 5/316; G06F 3/0484

(57) ABSTRACT

Conventional proprioceptive studies use commercial vibrators. Such vibrators have major drawbacks as their amplitude and frequency adjustments are limited or outside the range of frequency that stimulates the specific human biological sensors making them non-specific to each individual or muscle. At times, they are also expensive and not wearable. The presently disclosed subject matter relates to system and method for custom-developed vibrational systems having various adjustable and flexible parameters such as amplitude, frequency, delay time, and other user requirement functions that can work simultaneously and can be applied to different individuals and to different muscle spots. The presently disclosed system and method are robust, compact, wearable, economical, and easy to operate.

### PRIORITY CLAIM

The present application claims the benefit of priority of U.S. Provisional Patent Application No. 63/535,696, titled Vibrational System For Subject Proprioceptive Impairment Study, filed Aug. 31, 2023, and which is fully incorporated herein by reference for all purposes.

### BACKGROUND OF THE PRESENTLY DISCLOSED SUBJECT MATTER

The disclosure generally deals with medical device subject matter, and more specifically deals with proprioception vibrational systems and methods.

One of the key human feedback systems is the somatosensory system. Human postural control relies on three feedback systems: visual, vestibular, and somatosensory. The somatosensory system transmits proprioceptive, or body segment position sense, information from sensors in the skin, joints, tendons, and muscles. The nervous system utilizes this proprioceptive information to update a predictive model of where the body is in space, both relative to itself and the external environment.

Impaired proprioception has been established as an important factor predisposing individuals to injuries such as anterior cruciate ligament (ACL) rupture and lateral ankle ligament sprains. Proprioceptive deficits are also associated with, and hypothesized to be causally related to, chronic low back pain which is the leading cause of disability worldwide. While not an exhaustive list of the conditions impacted by impaired proprioception, ACL ruptures, lateral ankle ligament sprains, and chronic low back pain alone represent an enormous economic burden on the United States health care system and result in significant harm to the individuals afflicted.

Therefore, it is not surprising that researchers have sought to experimentally manipulate proprioception to elucidate the full scope of consequences associated with this impairment and to develop effective prevention and treatment strategies.

Literature dating back several decades demonstrates that vibration applied to muscles and tendons results in increased discharge from muscle proprioceptive sensors known as primary muscle spindle afferents. This results in altered proprioception and a kinesthetic illusion where individuals perceive that the vibrated muscle is lengthening when in fact, it is not. The ability to manipulate the proprioceptive feedback system is significant because it approximates an in vivo knockout model in humans.

To date, many study participants report no proprioceptive illusion (errors) at a fixed amplitude and frequency of the commercially available vibrators. It is clearly evident that different muscles in the human body and different individuals have different amplitude and frequency parameters of vibration needed that elicit a perceived proprioceptive error. Commercial vibrators are not structured for or adaptable for this purpose of human studies as they have a very limited frequency range with just a uniform single amplitude. This is a major drawback especially as we know different individuals and muscles require different parameters to create the proprioceptive error.

Thus, as noted, a major limitation of previous muscle vibration work (studies) is the application of standard vibration parameters for all participants when it is known that not everyone experiences proprioceptive impairment and kinesthetic illusion at the same vibration frequency and amplitude. Moreover, the frequencies and amplitudes used in previous investigations were based on studies that vibrated only arm muscles, despite known differences in structure and function between the muscles of the arm and the muscles of the trunk and lower extremities.

Commercially available products such as the Hypervolt (manufactured by Hyperice) and the Theragun (manufactured by Therabody) offer one to five discrete frequency settings with a uniform amplitude.

More specifically, Hypervolt (manufactured by Hyperice) provides only one to five discrete frequency settings with a uniform amplitude of vibration. Several big constraints/limitations of this device are: 1) the inability to control amplitude or frequency within the range of the biological sensor for different individuals and muscle locations; 2) not attachable to human subjects secondary to weight and structure; 3) parameters not in the range of biological sensors; and 4) components may interfere with other sensors needed to study human movement and the response to the kinesthetic illusion.

Theragun (manufactured by Therabody) also provides only one to five discrete frequency settings with a uniform amplitude of vibration. Several big constraints/limitations of this device are: 1) the inability to control amplitude or frequency within the range of the biological sensor for different individuals and muscle locations; 2) not attachable to human subjects secondary to weight and structure; 3) parameters not in the range of biological sensors; and 4) components may interfere with other sensors needed to study human movement and the response to the kinesthetic illusion.

Thus, such products are further limited because they function at amplitudes inconsistent with those used in previous literature inducing kinesthetic illusion and proprioceptive impairments. Researchers have used custom-made vibration systems that operate using a DC motor (like those manufactured by Maxon) which also only generates a discrete frequency and amplitude. This practice has prohibited the individualization of vibration parameters because Maxon-DC motor vibrational systems can only generate discrete frequencies and amplitudes, and are therefore restricted to working only on specific individuals who respond to the motor's specific parameters. Set frequency and amplitude motors potentially limit their use to response in only some individuals or specific muscles. Several big constraints/limitations of such device are: 1) the inability to control amplitude or frequency within the range of the biological sensor for different individuals and muscle locations; 2) not easily attachable to human subjects secondary to weight and structure; 3) parameters not in the range of biological sensors; and 4) components that may interfere with other sensors needed to study human movement and the response to the kinesthetic illusion.

Due to the lack of available vibration systems capable of generating the array of vibration parameters needed to personalize this stimulus, and the critical role of proprioception in preventing and ameliorating musculoskeletal diseases, it is clear that there is a gap in the current marketplace. It would greatly improve current research paradigms to have vibration systems that allow for finely tuned and personalized vibration parameters that can be adjusted for different muscle groups, body types, and experimental paradigms. A vibration system that accounts for the between-individual variation in response to vibration parameters would help increase scientific understanding of proprioceptive impairment and enable the creation of more effective prevention and treatment strategies to address this societal need.

### SUMMARY OF THE PRESENTLY DISCLOSED SUBJECT MATTER

Presently disclosed subject matter generally relates to improved proprioception vibrational systems and methods, and more particularly to innovative compact vibrational systems for performing trunk proprioceptive impairment studies.

Further, presently disclosed subject matter relates to addressing lower back pain, including disclosure of innovative compact vibrational systems with custom graphic user interface (GUI) for trunk proprioceptive impairment studies.

The presently disclosed systems and corresponding and/or associated methodologies help to address needs for modifiable vibrational systems which are portable, small in size to allow specific placement (unlike big vibrators), have minimal metal components, and are easy to fit to or attach to the human back or to be set to stimulate specific muscles/groups of muscles without stimulating all sensory receptors in the region. Additionally, the presently disclosed subject matter relates to a vibrational system that has customed or variable frequency and amplitude ranges where the user can manipulate both parameters (quantities) simultaneously based on the need. And further, presently the disclosed subject matter relates in part to a customized Graphical User Interface (GUI) for controlling the vibrational features like type of signal, amplitude, frequency, phase, delay time, channel, offset, etc. so that it enables changes to the parameters with ease of operation.

One exemplary embodiment disclosed herewith relates to a vibrational system for proprioceptive impairment studies. Such system preferably comprises a data acquisition processor selectively outputting a preamp level vibrational signal having user-determined parameters; a graphic user interface (GUI) for a user to provide designations to the data acquisition processor to determine parameters of the preamp level vibrational signal output by the data acquisition processor; an amplifier; and a vibrational tactor. Preferably, the amplifier is interconnected with the data acquisition processor for receiving the preamp level vibrational signal and outputting an amplified signal; and a vibrational tactor is for receiving the amplified signal from the amplifier while positioned on a selected area of a study subject, for conduct of a proprioceptive impairment study.

Another presently disclosed exemplary embodiment relates to a study system integrated into a single platform software, for inducing proprioceptive impairment for human sensorimotor studies of movement control and measuring temporary proprioceptive impairment on movement. Such a system preferably comprises a vibrational system as described herein, and at least one movement detection and measurement instrument such as an electrogoniometer for measuring joint position accuracy of a human study subject, an Electromyography (EMG) for monitoring muscle activities of a human study subject, and a force sensor and a load cell for pressure sensing and unidirectional force measuring of a human study subject, respectively. Such a study system allows a study user the ability to control the vibrational system allowing the select of waveforms, frequency, and amplitude of vibrations for use with different individuals and muscles in a study, requiring different parameters to create proprioceptive error.

It is to be understood that the presently disclosed subject matter equally relates to associated and/or corresponding methodologies. One exemplary such method relates to methodology for providing a vibrational system for use in a proprioceptive impairment study. Such exemplary methodology preferably comprises selectively outputting a preamp level vibrational signal having user-determined parameters; controllably amplifying the preamp level vibrational signal; and actuating a vibrational tactor with the amplified signal while the tactor is positioned on a selected area of a study subject, for the conduct of a proprioceptive impairment study.

Some such exemplary methodologies include further using a custom graphic user interface (GUI) to allow a user to adjust parameters of the preamp level vibrational signal, such parameters comprising one or more waveforms, amplitudes, phases, frequency, offsets, sampling rate delay time, type of signal, with such adjustments made as needed to elicit a perceived proprioceptive error in a particular study subject and for particular muscles of the study subject.

Other example aspects of the present disclosure are directed to systems, apparatus, tangible, non-transitory computer-readable media, user interfaces, memory devices, and electronic devices for ultrafast photovoltaic spectroscopy. To implement methodology and technology herewith, one or more processors may be provided, programmed to perform the steps and functions as called for by the presently disclosed subject matter, as will be understood by those of ordinary skill in the art.

Additional objects and advantages of the presently disclosed subject matter are set forth in, or will be apparent to, those of ordinary skill in the art from the detailed description herein. Also, it should be further appreciated that modifications and variations to the specifically illustrated, referred, and discussed features, elements, and steps hereof may be practiced in various embodiments, uses, and practices of the presently disclosed subject matter without departing from the spirit and scope of the subject matter. Variations may include, but are not limited to, substitution of equivalent means, features, or steps for those illustrated, referenced, or discussed, and the functional, operational, or positional reversal of various parts, features, steps, or the like.

Still further, it is to be understood that different embodiments, as well as different presently preferred embodiments, of the presently disclosed subject matter may include various combinations or configurations of presently disclosed features, steps, or elements, or their equivalents (including combinations of features, parts, or steps or configurations thereof not expressly shown in the figures or stated in the detailed description of such figures). Additional embodiments of the presently disclosed subject matter, not necessarily expressed in the summarized section, may include and incorporate various combinations of aspects of features, components, or steps referenced in the summarized objects above, and/or other features, components, or steps as otherwise discussed in this application. Those of ordinary skill in the art will better appreciate the features and aspects of such embodiments, and others, upon review of the remainder of the specification, and will appreciate that the presently disclosed subject matter applies equally to corresponding methodologies as associated with practice of any of the present exemplary devices, and vice versa.

These and other features, aspects and advantages of various embodiments will become better understood with reference to the following description and appended claims. The accompanying drawings, which are incorporated in and constitute a part of this specification, illustrate embodiments of the present disclosure and, together with the description, serve to explain the related principles.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1 illustrates a visual representation of an exemplary embodiment of a presently disclosed Custom Graphic User Interface (GUI) for an exemplary presently disclosed vibrational system;

FIG. 2(a) is an actual picture of an exemplary C2-HDLF linear actuator for use in the presently disclosed subject matter;

FIG. 2(b) is a table of the specifications of the exemplary C2-HDLF linear actuator of FIG. 2(a);

FIG. 2(c) graphically illustrates displacement vs. frequency curves of the exemplary C2-HDLF linear actuator of FIG. 2(a).

FIG. 3 illustrates images of an exemplary AK-170 audio amplifier for use in the presently disclosed subject matter;

FIG. 4 schematically represents all the exemplary individual components assembled to an exemplary final vibrational system in accordance with the present disclosure;

FIG. 5 graphically illustrates calibration curves of the presently disclosed vibrational system where the amplitudes in volts are converted to the displacements (average displacement in mm) at different frequency ranges;

FIG. 6 illustrates a schematic block diagram of components of an exemplary complete representative proprioceptive experimental impairment study arrangement, for incorporating devices in accordance with the presently disclosed subject matter;

FIG. 7 illustrates an exemplary block diagram of the circuit of the presently disclosed custom vibrational system software and its individual components;

FIG. 8 is an instantaneous snap real image of the circuit in the LabVIEW program and its details of the presently disclosed vibrational circuitry (inside of bold boxed areas) integrated with an exemplary force sensing circuit which is embedded in the overall complete circuit;

FIG. 9(a) is a close-up photograph of the subject presently disclosed vibrational system attached to a study subject as tested for proprioception;

FIG. 9(b) is a further photograph of the subject presently disclosed vibrational system attached to a study subject as shown in FIG. 9(a); and

FIG. 9(c) is a further photograph of the study subject of FIGS. 9(a) and 9(b), positioned relative to the study control and an exemplary embodiment of the presently disclosed subject matter, including a custom graphic user interface (GUI) pictured with a representative user.

Repeat use of reference characters in the present specification and drawings is intended to represent the same or analogous features, elements, or steps of the presently disclosed subject matter.

### DETAILED DESCRIPTION OF THE PRESENTLY DISCLOSED SUBJECT MATTER

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment.

In general, the present disclosure is directed to medical device related subject matter, and more specifically deals with proprioception vibrational systems and methods.

Human postural control relies on three feedback systems: visual, vestibular, and somatosensory. The somatosensory system transmits proprioceptive, or body segment position sense, information from sensors in the skin, joints, tendons, and muscles. The nervous system utilizes this proprioceptive information to update a predictive model of where the body is in space, both relative to itself and the external environment. Impaired proprioception has been established in the research literature as an important factor predisposing individuals to injuries such as anterior cruciate ligament (ACL) rupture and lateral ankle ligament sprains. Proprioceptive deficits are also associated with, and hypothesized to be causally related to, chronic low back pain which is the leading cause of disability worldwide. While not an exhaustive list of the conditions impacted by impaired proprioception, ACL ruptures, lateral ankle ligament sprains, and chronic low back pain alone represent an enormous economic burden on the United States health care system and result in significant harm to the individuals afflicted. Therefore, it is not surprising that researchers have sought to experimentally manipulate proprioception to elucidate the full scope of consequences associated with this impairment and to develop effective prevention and treatment strategies.

Literature dating back several decades demonstrates that vibration applied to muscles and tendons results in increased discharge from muscle proprioceptive sensors known as primary muscle spindle afferents. This results in altered proprioception and a kinesthetic illusion where individuals perceive that the vibrated muscle is lengthening when in fact, it is not. The ability to manipulate the proprioceptive feedback system is significant because it approximates an in vivo knockout model in humans. A major limitation of previous muscle vibration work (studies) is the application of standard vibration parameters for all participants when it is known that not everyone experiences proprioceptive impairment and kinesthetic illusion at a given vibration frequency and amplitude. Moreover, the frequencies and amplitudes used in previous investigations were based on studies that vibrated only arm muscles, despite known differences in structure and function between the muscles of the arm and the muscles of the trunk and lower extremities. Commercially available products such as the Hypervolt (manufactured by Hyperice) and the Theragun (manufactured by Therabody) offer one to five discrete frequency settings with a uniform amplitude. These products are further limited because they function at amplitudes inconsistent with those used in previous literature inducing kinesthetic illusion and proprioceptive impairments. Researchers have used custom made vibration systems that operate using a DC motor (like those manufactured by Maxon) which also only generates discrete frequencies and amplitudes. This practice has prohibited the individualization of vibration parameters.

Due to the lack of available vibration systems capable of generating the array of vibration parameters needed to personalize this stimulus, and the critical role of proprioception in preventing and ameliorating musculoskeletal diseases, it is clear that there is a gap in the current marketplace. It would greatly improve current research paradigms to have vibration systems that allow for finely tuned and personalized vibration parameters that can be adjusted for different muscle groups, body types, and experimental paradigms. A vibration system that accounts for the between-individual variation in response to vibration parameters would help increase scientific understanding of proprioceptive impairment and enable the creation of more effective prevention and treatment strategies to address this societal need.

Based on these facts, clearly there is a need for a vibrational system which is portable, accessible (unlike the big vibrators), minimal metal components, and easy to apply to the human back. Additionally, the most crucial feature required is a vibration system which has customed or variable frequencies and amplitude ranges where the user can manipulate both quantities simultaneously based on the need. Lastly, a customized Graphical User Interface (GUI) for controlling the vibrational features like type of signal, amplitude, frequency, phase, delay time, channel, offset, etc. so that it enables the quality of ease of operation.

Conventional proprioceptive studies use commercial vibrators. Such vibrators have major drawbacks as their amplitude and frequency adjustments are limited or outside the range of frequency that stimulates the specific human biological sensors making them non-specific to each individual or muscle. At times, they are also expensive and not wearable. The presently disclosed subject matter relates to system and method for custom-developed vibrational systems having various adjustable and flexible parameters such as amplitude, frequency, delay time, and other user requirement functions that can work simultaneously and can be applied to different individuals and to different muscle spots. The presently disclosed system and method are robust, compact, wearable, economical, and easy to operate.

Research has been performed in the areas of medical devices, proprioception, proprioception vibrational systems, flexible and wearable vibrational systems, and kinesthetic illusions. One upshot of such research was to show that the application of vibration to the muscles and tendons leads to increased discharge from muscle proprioceptive sensors.

To address the challenges described herein, we disclose a flexible, non-expensive, robust, compact, user-friendly vibrational device that has the flexibility of various features such as amplitude, frequency, phase, delay time, offset, type of waveform of the vibration. The vibrational system is also developed with custom Graphical User Interface (GUI) where even a layperson user can operate. The disclosed device subject matter basically breaks the constraints of the commercial vibrators enabling the ability not only to adapt parameters for different subjects but also different muscles using a single device saving huge costs and time. Additionally, the vibrational system is integrated with the other components of the proprioceptive impairment study which involves devices such as, electro goniometer, EMG, force sensors and load cell all in a single platform software making it more robust for proprioceptive studies.

Electromyography (EMG) measures muscle response or electrical activity in response to stimulation of the synergist or antagonist muscle. In addition to the foregoing, the EMG function requires the usage of minimal metal components in the vibrators because large metal components can interfere with EMG signals. Also, existing commercial vibrators are generally bulky and at times are not compatible and wearable in many body regions. Our presently disclosed innovative vibrational system uses almost 1-1.2 inches diameter of linear actuator (tactors) as a vibrational actuator which has minimal metal component, and it easily wearable (using medical tapes) without belts or application materials that can interfere with movement or enhance proprioception through other skin or joint sensors.

In other words, the device is superior in that it allows control of waveforms, frequency and amplitude of vibration making it more efficient, accurate, safe for use in human sensorimotor studies to enhance our understanding of movement control and the role of proprioceptive impairment on movement.

An exemplary embodiment of the presently disclosed subject matter may have as an overall vibrational system have primarily 4 major components: LabView custom GUI, NI-DAQ, Audio Amplifier, and Linear Vibrational Actuator.

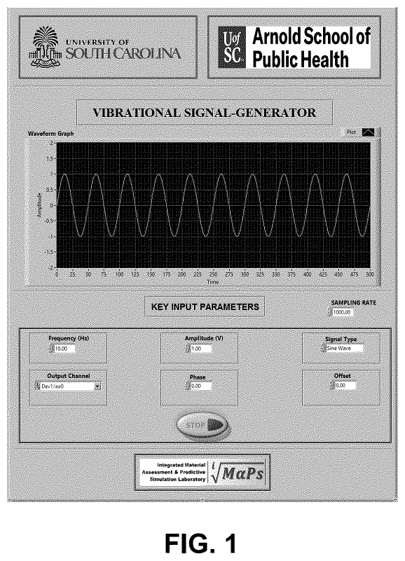

An initial aspect of the present disclosure was to first develop the subject vibrational device including the associated custom GUI based on an exemplary user's requirement. FIG. 1 is a visual representation of an exemplary embodiment of a presently disclosed Custom Graphic User Interface (GUI) for an exemplary presently disclosed vibrational system.

The presently disclosed exemplary overall system utilizes National Instruments (NI)-DAQ (data acquisition processor), which basically transmits output signal to the audio amplifier. The NI-DAQ system involves the software ‘LabVIEW’ which is utilized for developing the custom GUI. Based on user's different and novel functions required for the vibrational actuation, a custom GUI was developed. The GUI involves customed crucial parameters such as frequency, amplitude, type of signal, output channel for the DAQ, phase, offset, sampling rate, and delay time.

An additional component of the presently disclosed subject matter is the linear actuator. There are several linear actuators, vibrational devices, piezoelectric actuators, and many other systems available commercially for serving the sole purpose of the vibration. Per this present disclosure, we focused on the requirement of vibrational components that are small, lightweight and compact in addition to the capability of producing better vibrational output amplitude displacement. Other commercial conventional vibrational systems are in many instances very bulky, which typically either produces good amplitude, or they are smaller vibrators which produce less in terms of amplitudes.

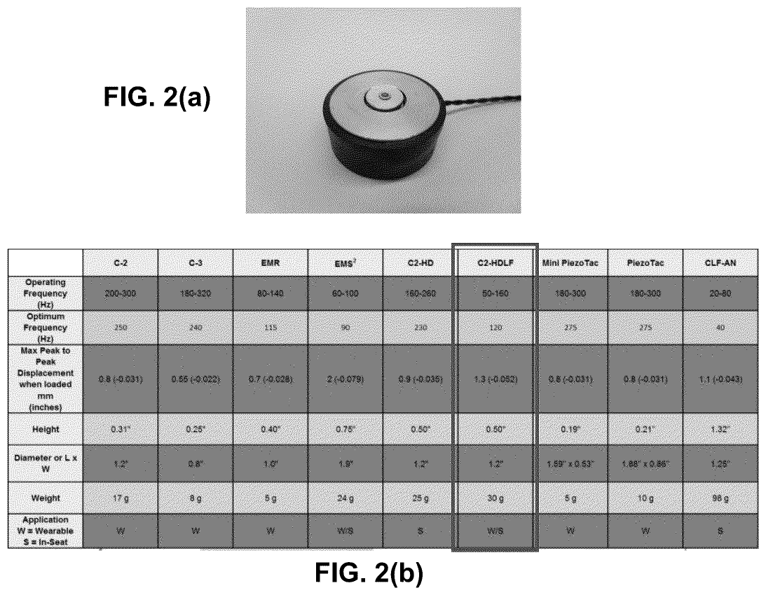

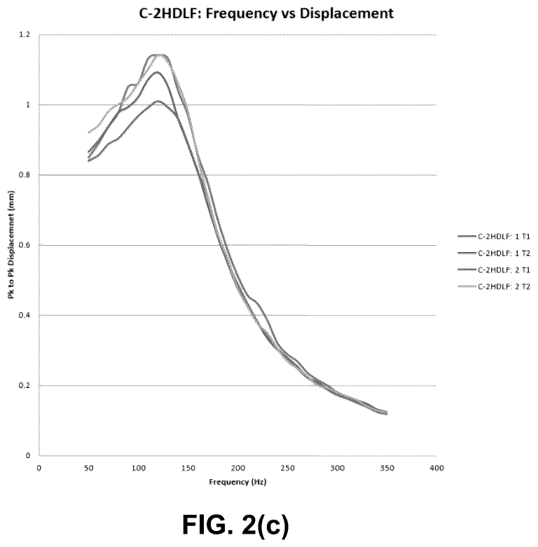

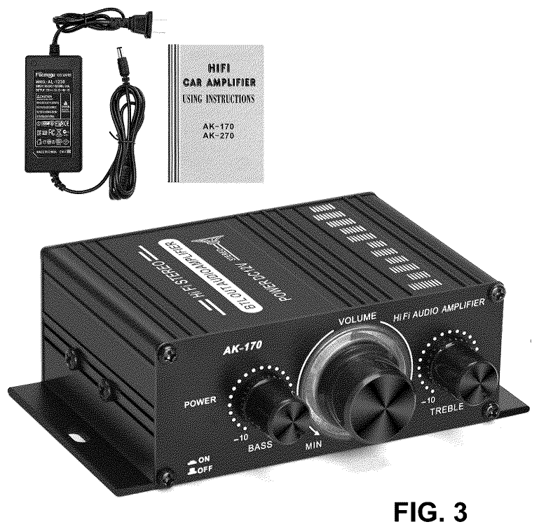

A significant focus was utilizing a pairable vibrational system with better vibrational output amplitudes and at the same time small and compact. The primary reason why small and compact vibrational systems are desirable is for being able to attach them to the lower back of the subject with some basic medical tapes and to avoid discomfort to the subject. As a result, we incorporated a C2-HDLF linear actuator. The operating frequency range falls under the optimal frequency range of the vibration where most of the subject feels and undergoes proprioception. FIG. 2(a) shows an actual picture of the exemplary C2-HDLF linear actuator for use in the presently disclosed subject matter, while FIG. 2(b) is a table of the specifications of the exemplary C2-HDLF linear actuator. FIG. 2(c) graphically illustrates displacement vs. frequency curves of the exemplary C2-HDLF linear actuator.

The exemplary linear actuator may be relatively small, almost 1.2 inches of diameter with almost 30 g weight. Such size and weight specifications result in a structure that is quite acceptable and user-friendly to wear.

The next component utilized in the exemplary system in accordance with the presently disclosed subject matter is the audio amplifier. As the output signal transmitted by the NI-DAQ has a very low power output, based on the linear actuator's optimal voltage range, a basic audio amplifier is utilized. An exemplary such component is a 20 W, 2 channels, AK-170 audio amplifier. FIG. 3 illustrates images of an exemplary AK-170 audio amplifier for use in the presently disclosed subject matter.

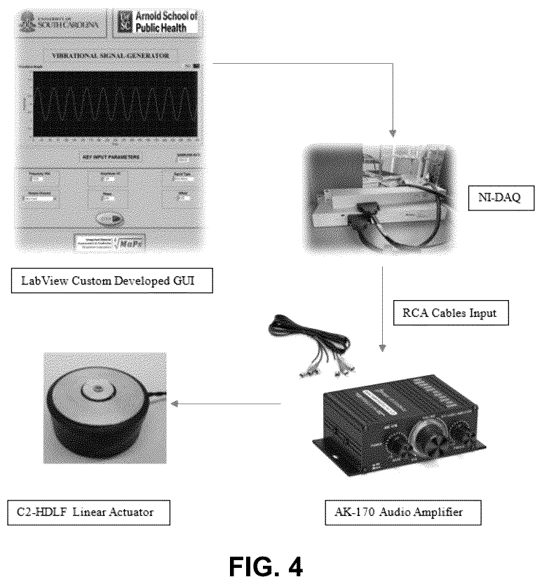

All of the above-referenced individual components are assembled together as a unit, for obtaining an exemplary embodiment of a final vibrational device in accordance with the present disclosure. FIG. 4 schematically represents all the exemplary individual components assembled into an exemplary final vibrational system in accordance with the present disclosure.

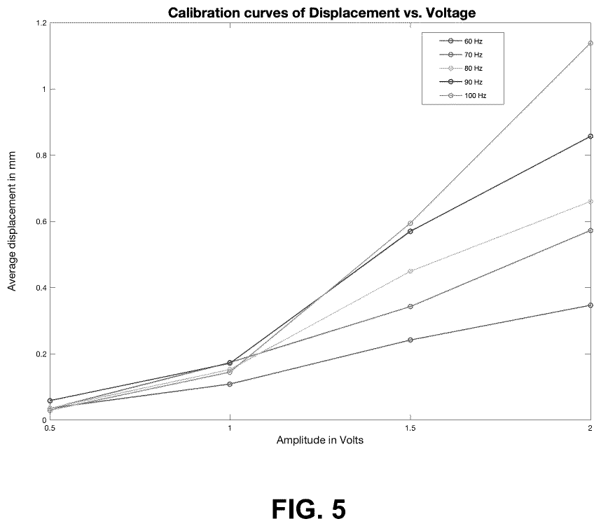

As a next step of our development of the presently disclosed subject matter, we analyzed the tactors and used displacement meters to convert the amplitudes from volts to millimeters. Based on the conversions, calibration curves were developed at potential different frequencies of interest such as 60-100 Hz. FIG. 5 graphically illustrates calibration curves of the presently disclosed vibrational system where the amplitudes in volts are converted to the displacements (average displacement in mm) at different frequency ranges.

Per the remainder of the present disclosure, we incorporate certain other devices that are used for the proprioception process. The devices that in some embodiments may be utilized are:
Vibration Actuator—The system that will produce vibrational output.
Force plate sensor—A force or a pressure sensor utilized for the spatial mapping of the trunk movement by a subject.
Electrogoniometer—A device that measures the joint position activities.
Electromyography (EMG)—A device that monitors muscle activation activities.
Load cell—A device that measures unidirectional force exerted by the trunk in the system.

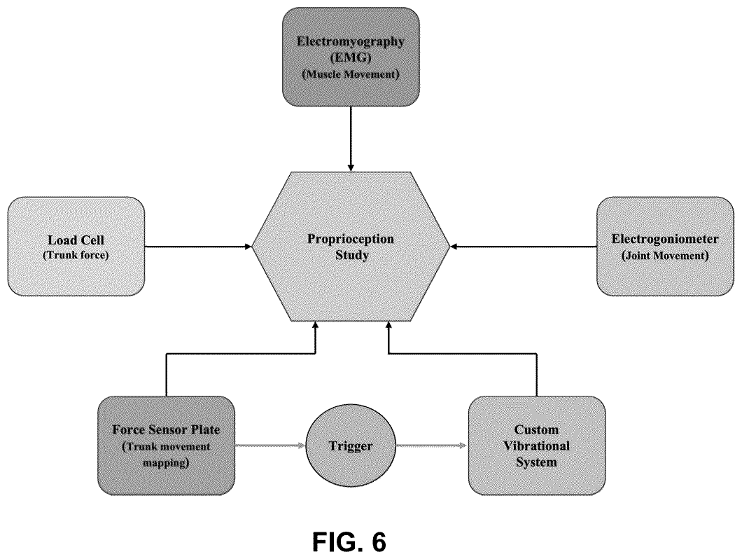

All these devices are specifically designed to interact and be controlled using the LabVIEW program in which we integrated the vibrational circuit. FIG. 6 illustrates a schematic block diagram of components of an exemplary complete representative proprioceptive experimental impairment study arrangement, for incorporating devices in accordance with the presently disclosed subject matter.

Additionally, FIG. 7 illustrates an exemplary block diagram of the circuit of the presently disclosed custom vibrational system software and its individual components.

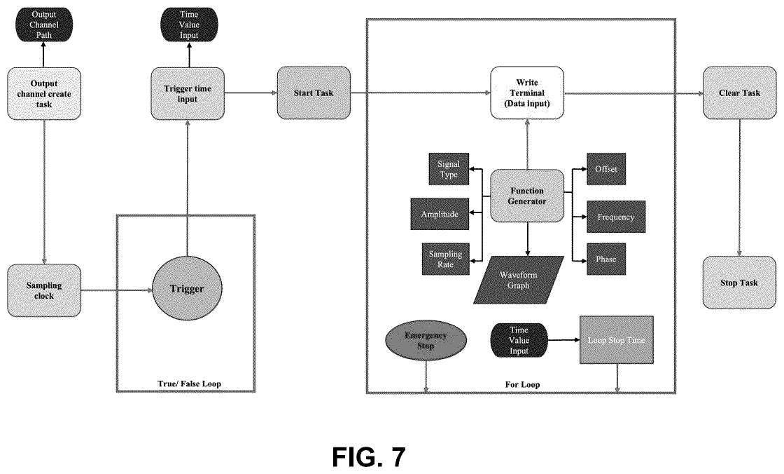

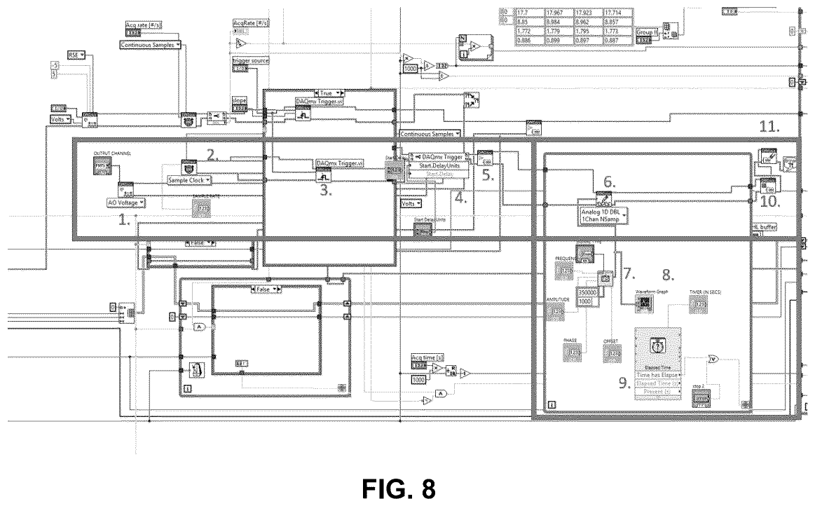

FIG. 8 is an instantaneous snap real image of the circuit in the LabVIEW program and its details of the presently disclosed vibrational circuitry (inside of bold boxed areas) integrated with an exemplary force sensing circuit which is embedded in the overall complete circuit.

The vibrational program was integrated with the main program circuitry at the trigger start interface. In the program, primarily 12 nodes along with two programming loops (if else and for loop) were applied to generate the vibrational signal output. The presently disclosed custom GUI was developed and customized later based on the developed features. The process below describes the flow of the vibrational program, with reference to eleven numbered aspects (nodes) of FIG. 8.

No. 1—DAQmx create channel—

This node basically creates the output channel to generate voltage signal for the vibrational system. The node is assigned with the output channel terminal where the user can specify the output port based on their requirement.

No. 2—DAQmx sample clock—

This node sets the source of the sample clock, the rate of the sample clock and the number of samples to acquire or generate. In this case we have connected it to the create channel node and assigned a sampling rate.

No. 3—DAQmx start trigger—

The next node is the trigger node which is built inside an if else loop. If the statement is true then upon pressing the external trigger button, the program executes and moves to the next node. This node primarily configures the task to start acquiring or generating samples on a rising or falling edge of a digital signal.

No. 4—DAQmx trigger—

This node executes or triggers the program to move to the next node after the trigger button is pressed. Here we assigned a delay timer which is basically the time lag in seconds after the trigger is pressed and the vibration actually starts. This feature has the flexibility of the user to assign any delay time value based on their convenience in secs.

No. 5—DAQmx start—

This node primarily transits the task to the running state to begin the acquisition or generation of the signals. Once the delay timed is passed after the trigger feature, the start feature starts the program and proceeds further.

No. 6—DAQmx write—

This node writes one or more floating-point samples to a task that consists of single analog output channel. This node is contained inside a for loop. This write node consist of a data input to which the function generator node is connected.

No. 7—Basic function generator node—

The basic function generator is a node that sends signals to the write function. The function generator consists of different waveforms along with different signal parameters such as amplitude, phase, frequency, offset, phase, and signal type. The user can select different parameters which a novelty compared to the conventional vibrational systems and set based on their convenience.

No. 8—Waveform graph—

This node enables in a GUI to show the waveform generated based on the signal provided. With the help of this node, the user can monitor the waveform graph visually.

No. 9—Elapsed time—

As mentioned earlier, the DAQmx write is inside a for loop, and it iterates to the time specified based on this elapsed time feature. Based on the user requirement, target time is set and the for loop runs for that specified time and it gives the versatility to control the vibration autonomously based to the elapsed time specified in the GUI.

No. 10—DAQmx stop—

Once the program exits the for loop, it proceeds to the DAQmx stop node where the task is stopped, and it returns to the task as it was before the DAQmx start.

No. 11—DAQmx clear task—

This feature basically clears the task and aborts the tasks, if necessary and releases any resources that the task reserved.

Initial utilization was to simulate proprioception impairment by stimulating muscle spindles and creating an illusion of muscle lengthening (brain perception) and assess the changes to movement control and proprioception tasks on different subjects. Thus, pilot testing of the device was conducted on five human subjects to test the device application to trunk extensor muscles, safety, determine any interference with other biomechanical sensors, and the participants' response to specific stimulation frequency/amplitude that elicit the proprioception illusion.

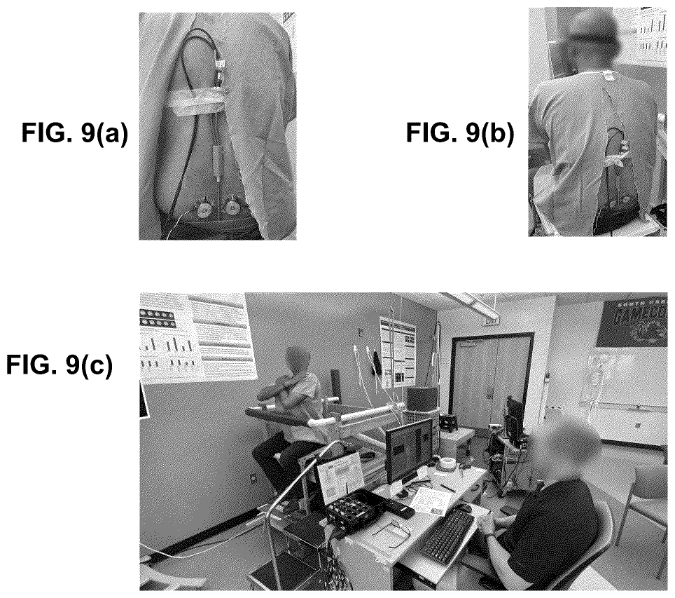

The presently disclosed technology has already been deployed and tested on a number of subjects for development of the protocol for proprioceptive impairments study, such as in arrangements, for example, as represented by FIGS. 9(a)-9(c), which show pictures of vibrating tactors (linear actuators) as attached to a subject in the lower back region.

FIGS. 9(a)-9(c) illustrate a presently disclosed vibrational system along with an exemplary electro goniometer attached to a subject for study, to be tested for the proprioception on top of a force sensing plate for the trunk movement. The subject was blindfolded and was tested for the proprioception study with cross hands posture. Various trunk movements (specific to physiological aspects) were tested, and comparisons were made with and without the vibrational actuation. More particularly, FIG. 9(a) is a close-up photograph of the subject presently disclosed vibrational system attached to a study subject as tested for impaired proprioception. FIG. 9(b) is a further photograph of the subject presently disclosed vibrational system attached to a study subject as shown in FIG. 9(a). FIG. 9(c) is a further photograph of the study subject of FIGS. 9(a) and 9(b), positioned relative to the study control and an exemplary embodiment of the presently disclosed subject matter, including a custom graphic user interface (GUI) pictured with a representative user.

Currently, the system has shown good and reproducible results. If used on a large scale, the presently disclosed device will not only be used as a proprioceptive study device but will be used in the biomedical industry, pain management, and medical devices. Due to its ease of operation and inexpensive nature, it is expected to be convenient for use by technical and non-technical personnel.

While certain embodiments of the disclosed subject matter have been described using specific terms, such description is for illustrative purposes only, and it is to be understood that changes and variations may be made without departing from the spirit or scope of the subject matter.

What is claimed is:

1. A vibrational system for proprioceptive impairment study, comprising:
a data acquisition processor selectively outputting a preamp level vibrational signal having user-determined parameters;
a graphic user interface (GUI) for a user to provide designations to the data acquisition processor to determine parameters of the preamp level vibrational signal output by the data acquisition processor;
an amplifier interconnected with the data acquisition processor for receiving the preamp level vibrational signal and outputting an amplified signal; and
a vibrational tactor for receiving the amplified signal from the amplifier while positioned on a selected area of a study subject, for conduct of a proprioceptive impairment study.

2. The vibrational system according to claim 1, wherein the GUI comprises a custom GUI that allows a user to adjust parameters comprising one or more of waveform, amplitude, phase, frequency, offset, phase, sampling rate delay time, type of signal, and processor output channel of the preamp level vibrational signal output.

3. The vibrational system according to claim 1, wherein the GUI comprises a custom GUI which allows a user to adjust amplitude and frequency parameters of vibration needed to elicit a perceived proprioceptive error in a particular study subject and for particular muscles of the study subject.

4. The vibrational system according to claim 1, wherein the GUI comprises a custom GUI which allows a user to fine tune and personalize vibration parameters adjusted for different muscle groups, body types, and experimental paradigms.

5. The vibrational system according to claim 1, wherein the vibrational tactor comprises one or more of a linear actuator, a piezoelectric actuator, or other controllable vibrational device.

6. The vibrational system according to claim 1, wherein the amplifier comprises an audio amplifier.

7. The vibrational system according to claim 6, wherein the audio amplifier has a power output rating of at least 20 W, and has at least 2 channels.

8. The vibrational system according to claim 1, wherein the GUI comprises a custom GUI and the processor comprises a LabVIEW-enabled programmable processor.

9. The vibrational system according to claim 1, further comprising:
a plurality of respective actuators for receiving a respective amplified signal from the amplifier while positioned on respective selected areas of a corresponding plurality of study subjects;
wherein the amplifier comprises an audio amplifier having at least a corresponding plurality of channels; and
the data acquisition processor selectively outputs a plurality of respective preamp level vibrational signals having respective user-determined parameters, so that selected vibrational interactions can simultaneously be applied to at least one of different study subject individuals and to different muscles.

10. The vibrational system according to claim 1, wherein the tactor comprises a linear actuator having a diameter of no larger than about 1.2 inches, and a weight of no more than about 30 grams, adapted for being worn on a subject's lower back.

11. The vibrational system according to claim 1, wherein the GUI comprises a custom GUI which allows a user to adjust frequency in a range from 60 to 100 Hz.

12. A study system integrated in a single platform software, for conducting proprioceptive impairment study for human sensorimotor studies of movement control and proprioceptive impairment on movement, comprising:
a vibrational system according to claim 1; and
at least one of an electrogoniometer for measuring joint position of a human study subject, an Electromyography (EMG) for monitoring muscle activity of a human study subject, and a force sensor and a load cell for pressure sensing and unidirectional force measuring of a human study subject;
wherein the study system allows a study user to use the vibrational system to control waveforms, frequency, and amplitude of vibrations for use with different individuals and muscles in a study, requiring different parameters to create proprioceptive error.

13. A methodology for providing a vibrational system for use in a proprioceptive impairment study, comprising:
selectively outputting a preamp level vibrational signal having user-determined parameters;
controllably amplifying the preamp level vibrational signal; and
actuating a vibrational tactor with the amplified signal while the tactor is positioned on a selected area of a study subject, for conduct of a proprioceptive impairment study.

14. The methodology according to claim 13, further comprising using a custom graphic user interface (GUI) to allow a user to adjust parameters of the preamp level vibrational signal, such parameters comprising one or more of waveform, amplitude, phase, frequency, offset, phase, sampling rate delay time, type of signal, with such adjustments made as needed to elicit a perceived proprioceptive error in a particular study subject and for particular muscles of the study subject.

15. The methodology according to claim 14, further comprising using the custom GUI to allow a user to fine tune and personalize vibration parameters adjusted for different muscle groups, body types, and experimental paradigms.

16. The methodology according to claim 13, wherein the vibrational tactor comprises one or more of a linear actuator, a piezoelectric actuator, or other controllable vibrational device.

17. The methodology according to claim 13, wherein controllably amplifying comprises using an audio amplifier.

18. The methodology according to claim 17, wherein the audio amplifier has a power output rating of at least 20 W, and has at least 2 channels.

19. The methodology according to claim 14, further comprising using the custom GUI for inputting parameters into a data acquisition programmable processor.

20. The methodology according to claim 14, further comprising providing a plurality of respective actuators for receiving a respective amplified signal while positioned on respective selected areas of a corresponding plurality of study subjects, with each respective amplified signal having respective user-determined parameters, so that selected vibrational interactions can simultaneously be applied to at least one of different study subject individuals and to different muscle spots.

21. The methodology according to claim 13, wherein the tactor comprises a linear actuator having a diameter of no larger than about 1.2 inches, and a weight of no more than about 30 grams, adapted for being worn on a subject's lower back.

22. A study system methodology integrated in a single platform software, for conducting proprioceptive impairment study for human sensorimotor studies of movement control and proprioceptive impairment on movement, comprising:
using a methodology for providing a vibrational system according to claim 13, together with using at least one of an electrogoniometer for measuring joint position activities of a human study subject, an Electromyography (EMG) for monitoring muscle movement activities of a human study subject, and a force sensor and a load cell for pressure sensing and unidirectional force measuring of a human study subject;
wherein the study system methodology allows a study user to control waveforms, frequency, and amplitude of vibrations for use with different individuals and muscles in a study requiring different parameters to create proprioceptive error for different individuals in the study.
